(19) United States
(12) Patent Application Publication
(10) Pub. No.: US 2004/0132697 A1
(43) Pub. Date: Jul. 8, 2004

(54) Treatment of female sexual dysfunction

(75) Inventors: Richard James Thurlow, Sandwich (GB); Christopher Peter Wayman, Sandwich (GB)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/699,602

(22) Filed: Oct. 31, 2003

Related U.S. Application Data
(60) Provisional application No. 60/429,729, filed on Nov. 27, 2002.

(30) Foreign Application Priority Data
Nov. 6, 2002 (GB) 0225908.3

(51) Int. Cl. A61K 31/517; A61K 31/56

(52) U.S. Cl. 514/169; 514/266.1

(57) ABSTRACT

The present invention relates the use of α_{1A} and/or α_{1L} adrenergic receptor antagonists for the treatment of female sexual dysfunction (FSD), in particular female sexual arousal disorder (FSAD) and/or female orgasmic disorder (FOD). The present invention also relates to a method of treatment of FSD, in particular FSAD and/or FOD, as well as to assays to screen for compounds useful in the treatment of FSD, in particular FSAD and/or FOD.

This application claims priority to U.S. Provisional Serial No. 60/429,729, filed Nov. 27, 2002, which claims priority to GB Application Serial No. 0225908.3, filed Nov. 6, 2002.

### FIELD OF INVENTION

The present invention relates to the use of α_{1A} and/or α_{1L} adrenergic receptor antagonists for the treatment of female sexual dysfunction (FSD), in particular female sexual arousal disorder (FSAD) and/or female orgasmic disorder (FOD).

The present invention also relates to a method of treatment of FSD, in particular FSAD and/or FOD.

The present invention also relates to assays to screen for compounds useful in the treatment of FSD, in particular FSAD and/or FOD.

Female Sexual Response

The female sexual response phase of arousal is not easily distinguished from the phase of desire until physiological changes begin to take place in the vagina and clitoris as well as other sexual organs. Sexual excitement and pleasure are accompanied by a combination of vascular and neuromuscular events which lead to engorgement of the clitoris, labia and vaginal wall, increased vaginal lubrication and dilatation of the vaginal lumen (Levin,R. J. (1980) Clin. Obstet. Gynecol. 7, 213-252; Ottesen, B. et al (1983) Eur. J. Clin. Invest. 13, 321-324; Levin, R. J. (1991) Exp. Clin. Endocrinol. 98, 61-69; Levin, R. J. (1992) Annu. Rev. Sex Res. 3, 1-48; Sjoberg,I (1992) Acta Obst. Gynecol. Scand 71, 84-85; Wagner, G. (1992) Sem. Neurol 12, 87-97; Schiavi et al. (1995) Psychiat. Clin. North Am. 18, 7-23; Berman, J. R. et al. (1999) Urology 54, 385-391).

Vaginal engorgement enables transudation to occur and this process is responsible for increased vaginal lubrication. Transudation allows a flow of plasma through the epithelium and onto the vaginal surface, the driving force for which is increased blood flow in the vaginal capillary bed during the aroused state. In addition engorgement leads to an increase in vaginal length and luminal diameter, especially in the distal ⅔ of the vaginal canal. The luminal dilatation of the vagina is due to a combination of smooth muscle relaxation of its wall and skeletal muscle relaxation of the pelvic floor muscles. Some sexual pain disorders such as vaginismus are thought to be due, at least in part, to inadequate relaxation preventing dilatation of the vagina; it has yet to be ascertained if this is primarily a smooth or skeletal muscle problem.

The vasculature and micro vasculature of the vagina are innervated by nerves containing neuropeptides and other neurotransmitter candidates. These include calcitonin gene-related peptide (CGRP), neuropeptide Y (NPY), nitric oxide synthase (NOS), substance P and vasoactive intestinal peptide (VIP) (Hoyle, C. H. V. et al. (1996) J. Anat. 188, 633-644). Peptides that are present in the clitoris are discussed infra. Nitric oxide synthase, which is often colocalised with VIP, displays a greater expression, immunologically, in the deep arteries and veins rather than in the blood vessels of the propria (Hoyle, C. H. V. et al. (1996) J. Anat. 188, 633-644).

Female Sexual Dysfunction

It is known that some individuals can suffer from female sexual dysfunction (FSD). Studies investigating sexual dysfunction in couples reveals that up to 76% of women have complaints of sexual dysfunction and that 30-50% of women in the USA experience FSD.

FSD is best defined as the difficulty or inability of a woman to find satisfaction in sexual expression. FSD is a collective term for several diverse female sexual disorders (Basson et al (2000) J. Urol. 163, 888-893). The woman may have lack of desire, difficulty with arousal or orgasm, pain with intercourse or a combination of these problems. Several types of disease, medications, injuries or psychological problems can cause FSD. Sub-types of FSD include hypoactive sexual desire disorder, female sexual arousal disorder (FSAD), female orgasmic disorder (FOD) and sexual desire disorder.

Treatments in development are targeted to treat specific subtypes of FSD, predominantly desire and arousal disorders.

The categories of FSD are best defined by contrasting them to the phases of normal female sexual response: desire, arousal and orgasm (Leiblum, S. R. (1998) Int. J. Impotence Res. 10, S104-S106). Desire or libido is the drive for sexual expression—and manifestations often include sexual thoughts either when in the company of an interested partner or when exposed to other erotic stimuli. In contrast, sexual arousal is the vascular response to sexual stimulation, an important component of which is vaginal lubrication and elongation of the vagina. Thus, sexual arousal, in contrast to sexual desire, is a response relating to genital (e.g. vaginal and clitoral) blood flow and not necessarily sensitivity. Orgasm is the release of sexual tension that has culminated during arousal. Hence, FSD typically occurs when a woman has an inadequate or unsatisfactory response in any of these phases, usually desire, arousal or orgasm. FSD categories include hypoactive sexual desire disorder, sexual arousal disorder, orgasmic disorders and sexual pain disorders.

Hypoactive sexual desire disorder is present if a woman has no or little desire to be sexual, and has no or few sexual thoughts or fantasies. This type of FSD can be caused by low testosterone levels, due either to natural menopause or to surgical menopause. Other causes include illness, medications, fatigue, depression and anxiety.

Female sexual arousal disorder (FSAD) is characterised by inadequate genital response to sexual stimulation. The genitalia (e.g. the vagina and/or the clitoris) do not undergo the engorgement that characterises normal sexual-arousal. The vaginal walls are poorly lubricated, so that intercourse is painful. Orgasms may be impeded. Arousal disorder can be caused by reduced oestrogen at menopause or after childbirth and during lactation, as well as by illnesses, with vascular components such as diabetes and atherosclerosis. Other causes result from treatment with diuretics, antihistamines, antidepressants eg SSRIs or antihypertensive agents. FSAD is discussed in more detail infra.

Sexual pain disorders (which include dyspareunia and vaginismus) are characterised by pain resulting from penetration and may be caused by medications which reduce lubrication, endometriosis, pelvic inflammatory disease, inflammatory bowel disease or urinary tract problems.

The prevalence of FSD is difficult to gauge because the term covers several types of problem, some of which are difficult to measure, and because the interest in treating FSD is relatively recent. Many women's sexual problems are associated either directly with the female ageing process or with chronic illnesses such as diabetes and hypertension. Numerous studies have also shown that between 11-48% of women overall may have reduced sexual desire with age. Similarly, between 11-50% of women report problems with arousal and lubrication, and therefore experience pain with intercourse. Vaginismus is far less common, affecting approximately 1% of women.

Because FSD consists of several subtypes that express symptoms in separate phases of the sexual response cycle, there is not a single therapy. Current treatment of FSD focuses principally on psychological or relationship issues. Treatment of FSD is gradually evolving as more clinical and basic science studies are dedicated to the investigation of this medical problem. Female sexual complaints are not all psychological in pathophysiology, especially for those individuals who may have a component of vasculogenic dysfunction (eg FSAD) contributing to the overall female sexual complaint. There are at present no drugs licensed for the treatment of FSD. Empirical drug therapy includes oestrogen administration (topically or as hormone replacement therapy), androgens or mood-altering drugs such as buspirone or trazodone. These treatment options are often unsatisfactory due to low efficacy or unacceptable side effects.

Since interest is relatively recent in treating FSD pharmacologically, therapy consists of the following: psychological counselling, over-the-counter sexual lubricants, and investigational candidates, including drugs approved for other conditions. These medications consist of hormonal agents, either testosterone or combinations of oestrogen and testosterone and more recently vascular drugs, that have proved effective in male erectile dysfunction. None of these agents has been approved for the treatment of FSD.

Female Sexual Arousal Disorder (FSAD)

The sexual arousal response consists of vasocongestion in the pelvis, vaginal lubrication and expansion and swelling of the external genitalia. The disorder causes marked distress and/or interpersonal difficulty. Studies investigating sexual dysfunction in couples reveals that there is a large number of females who suffer from sexual arousal dysfunction; otherwise known as female sexual arousal disorder (FSAD).

The Diagnostic and Statistical Manual (DSM) IV of the American Psychiatric Association defines Female Sexual Arousal Disorder (FSAD) as being: “a persistent or recurrent inability to attain or to maintain until completion of the sexual activity adequate lubrication-swelling response of sexual excitement. The disturbance must cause marked distress or interpersonal difficulty.”

FSAD is a highly prevalent sexual disorder affecting pre-, per- and post-menopausal women, whether or not they were treated with hormone replacement therapy (±HRT). It is associated with concomitant disorders such as depression, cardiovascular diseases, diabetes and UG disorders.

The primary consequences of FSAD are lack of engorgement/swelling, lack of lubrication and lack of pleasurable genital sensation. The secondary consequences of FSAD are reduced sexual desire, pain during intercourse and difficulty in achieving an orgasm.

It has recently been hypothesised that there is a vascular basis for at least a proportion of patients with symptoms of FSAD (Goldstein, I & Berman, J. R (1998) Int. J. Impotence Res. 10, S84-S90) with animal data supporting this view (Park, K. et al. (1997) Int. J. Impotence Res. 9, 27-37).

Drug candidates for treating FSAD, which are under investigation for efficacy, are primarily erectile dysfunction therapies that promote circulation to the male genitalia. They consist of two types of formulation, oral or sublingual medications (Apomorphine, Phentolamine, Sildenafil), and prostaglandin (PGE₁—Alprostadil) that are injected or administered transurethrally in men, and topically to the genitalia in women.

Phentolamine mesylate is a combined α₁ and α₂ adrenergic receptor antagonist, which was originally approved for the treatment of pheochromocytoma-induced hypertension and norepinephrine-related dermal necrosis. Since the early 1980s, it has been used in combination with other agents for intracavernosal injection therapy of erectile dysfunction, and more recently, an oral formulation of phentolamine was developed for treatment of mild or psychogenic erectile dysfunction. A small pilot study showed that the drug appeared to lead to mild improvements in FSAD (Rosen, R. C. et al (1999) J. Sex Marital Therapy 25, 137-144). A recent study identified that functional α₁ and α₂ adrenergic receptors are expressed on rabbit vagina (Kim et al (2002) Life Sciences 71, 2909-2920), and demonstrated that a adrenergic receptor antagonists can increase blood flow to the vagina and therefore may have potential as pharmacotherapeutic agents in treating some symptoms associated with FSAD.

There are three distinct α₂ adrenergic receptor subtypes, called α_{2A}, α_{2B}, and α_{2C}, which generally have a critical role in regulating neurotransmitter release from sympathetic nerves and from adrenergic neurons in the central nervous system.

A main function of α₁-adrenergic receptors (α₁ receptors) is to activate mitogenic responses and regulate growth and proliferation of many cells as well as being involved in mediating the contraction of vascular smooth muscle. There are 3 cloned α₁ receptor subtypes, α_{1A}, α_{1B}, and α_{1D} (Schwinn D. A. et al (1995) J. Pharmacol. Exp. Ther. 272, 134-142), all of which signal through the G_{q/11} family of G-proteins, and different subtypes show different patterns of activation.

Although cloning studies suggest that all subtypes of α₁ receptors have been identified at the molecular level, it has been demonstrated that the α₁ receptor subtype mainly found in human prostate displays a different pharmacology to the cloned subtypes, and the receptor has been termed α_{1L} to reflect this fact. Tissue distribution studies of the cloned α₁ receptor subtypes suggest that α_{1A} is expressed in the human prostate, and the assumption is therefore that the α_{1L} receptor corresponds to α_{1A} molecularly, but is a different receptor at the functional level, possibly influenced by other factors (for example, the lipid composition in the membrane, or formation of complexes with other proteins or itself) to display the α_{1L} pharmacology (Daniels, D. V. et al (1999) Eur. J. Pharmacol. 370, 337-343).

Surprisingly, we have found that α_{1A} and/or α_{1L} antagonists, preferably selective α_{1A} and/or α_{1L} antagonists, originally developed for treatment of benign prostatic hyperplasia (BPH), are also advantageous in treating FSD, preferably FSAD and/or FOD. In addition to increasing vaginal blood flow they may also restore sexual arousal, increase vaginal lubrication, enhance vaginal and clittoral sensitivity, and therefore enhance orgasmic function.

The present invention therefore seeks to provide an effective means of treating FSD, and in particular FSAD and/or FOD, by using α_{1A} or α_{1L} antagonists, preferably selective α_{1A} or α_{1L} antagonists, alone or in combination with other agents.

Aspects of the Invention

A seminal finding of the present invention is the ability to treat a female suffering from female sexual dysfunction (FSD), preferably female sexual arrousal disorder (FSAD) and/or female orgasmic disorder (FOD), with an antagonist for α_{1A} and/or α_{1L} adrenergic receptors. If the female to be treated is postmenopausal, she will preferably be on hormone replacement therapy (HRT), even more preferably she will also receive androgen therapy.

Therefore the invention relates to α_{1A} and/or α_{1L} receptor antagonists for use in the treatment of FSD, preferably FSAD and/or FOD. The invention also relates to the use of α_{1A} and/or α_{1L} adrenergic receptor antagonists for the manufacture of a medicament for the treatment of FSD, preferably FSAD and/or FOD. The invention also relates to a method of treatment of FSD, preferably FSAD and/or FOD, with an α_{1A} and/or α_{1L} adrenergic receptor antagonist. One aspect of the invention is therefore a method of treating FSD, preferably FSAD and/or FOD, comprising the administration to a patient in need of such treatment of an effective amount of an α_{1A} or an α_{1L} antagonist.

The α_{1A} and/or α_{1L} adrenergic receptor antagonists preferably will have a Kᵢ in a binding assay of less than 100 nM, more preferably a Kᵢ of less than 10 nM, even more preferably a Kᵢ of less than 1 nM. The Kᵢ may be measured in a binding assay (see, for example, Example 2). The α_{1A} and/or α_{1L} adrenergic receptor antagonists preferably will have a pA₂ in a functional assay of greater than 7, preferably a pA₂ of greater than 8, most preferably a pA₂ of greater than 9. The pA₂ may be measured in a functional assay, measuring contractile responses in rabbit aorta or human prostate for α_{1L} receptors, or rat vas deferens for α_{1A} receptors (see, for example, Example 3).

Preferably the α_{1A} and/or α_{1L} adrenergic receptor antagonists will be at least 10 fold selective over α_{1B}, more preferably at least 100 fold selective over α_{1B}. Preferably the α_{1A} and/or α_{1L} adrenergic receptor antagonists will be at least 10 fold selective over α_{1D}, more preferably at least 100 fold selective over α_{1D}. More preferably, the α_{1A} and/or α_{1L} adrenergic receptor antagonists will be at least 10 fold selective over α_{1B} and at least 10 fold selective over α_{1D}, most preferably at least 100 fold selective over α_{1B} and at least 100 fold selective over α_{1D}.

Suitable α_{1A} and/or α_{1L} antagonists include a compound of formula (I), as disclosed in international application number PCT/IB03/00998:

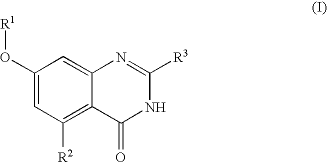

or a pharmaceutically acceptable salt or solvate thereof, wherein

R¹ represents C₁₋₄ alkyl;

R² represents halo, C₁₋₄ alkyl, C₃₋₆ cycloalkyl, C₃₋₆ cycloalkyloxy, —SO₂(C₁₋₄ alkyl), C₁₋₄ alkyloxy (optionally substituted by C₃-C₆ cycloalkyl or C₁-C₄ alkoxy), Het or —OHet;

R³ represents a bicyclic group of the formula

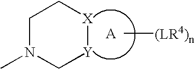

wherein X and Y are selected from C and N, provided that at least one is C;

Ring A together with X and Y represents a 5- or 6-membered aromatic ring containing 0, 1, 2 or 3 nitrogen atoms in the ring;

n is 0, 1 or 2

L independently represents a direct link, C₁₋₄ alkylene or C₁₋₄ alkoxyalkylene;

R⁴ independently represents H, —NR⁵R⁶, C₃₋₆ cycloalkyl, —OR⁷, Het¹ or Het⁴;

R⁵ and R⁶ are independently selected from H, C₃₋₆ cycloalkyl, C₃₋₆ cycloalkyl-C₁₋₄ alkylene, —SO₂(C₁₋₄ alkyl) and C₁₋₄ alkyl (optionally substituted with —OR⁸, —NR¹⁰R¹¹, Het¹ or Het⁴);

R⁷ is selected from H, C₁₋₄ alkyl, C₁₋₄ alkoxyalkyl, C₃₋₆cycloalkyl, Het² and C₁₋₄alkyl-Het³;

R⁸ is H or C₁₋₄ alkyl;

Het, Het¹, Het² and Het³ independently represent a 4 to 7 membered saturated heterocyclic group which may be mono- or bi-cyclic and which contains one or more heteroatoms selected from N, O or S, optionally substituted with OR⁹ and/or C₁₋₄ alkyl optionally substituted by OR⁹;

Het⁴ represents a 5 or 6 membered unsaturated heterocyclic group containing one or more heteroatoms selected from N, O or S, optionally substituted with C₁₋₄ alkyl;

R⁹ is H or C₁₋₄ alkyl;

R¹⁰ and R¹¹ are independently selected from H and C₁₋₄ alkyl.

Preferably, suitable α_{1A} and/or α_{1L} antagonists are compounds exemplified in international patent application PCT/IB03/00998, more preferably one of the following compounds:

5-cyclopropyl-7-methoxy-2-(2-([dimethylamino]methyl)-7,8-dihydro[1,6]naphthyridin-6(5H)-yl)-4(3H)-quinazolinone;

5-cyclopropyl-7-methoxy-2-(2-(1-pyrrolidinylmethyl)-7,8-dihydro[1,6]naphthyridin-6(5H)-yl)-4(3H)-quinazolinone;

5-cyclopropyl-7-methoxy-2-(2-(4-methoxypiperidin-1-ylmethyl)-7,8-dihydro[1,6]naphthyridin-6(5H)-yl)-4(3H)-quinazolinone;

5-cyclopropyl-7-methoxy-2-(2-(4-morpholinylmethyl)-7,8-dihydro[1,6]naphthyridin-6(5H)-yl)-4(3H)-quinazolinone;

5-cyclopropyl-7-methoxy-2-(5-([dimethylamino]methyl)-3,4-dihydro[2,6]naphthyridin-2(1H)-yl)-4(3H)-quinazolinone;

5-cyclopropyl-7-methoxy-2-(5-(1-pyrrolidinylmethyl)-3,4-dihydro[2,6]naphthyridin-2(1H)-yl)-4(3H)-quinazolinone;

5-cyclopropyl-7-methoxy-2-(5-(1-piperidinylmethyl)-3,4-dihydro[2,6]naphthyridin-2(1H)-yl)-4(3H)-quinazolinone;

5-cyclopropyl-7-methoxy-2-(5-(4-morpholinylmethyl)-3,4-dihydro[2,6]naphthyridin-2(1H)-yl)-4(3H)-quinazolinone;

5-cyclopropyl-7-methoxy-2-(5-[(1S,4S)-2-oxa-5-azabicyclo[2.2.1]hept-5-ylmethyl]-3,4-dihydro[2,6]naphthyridin-2(1H)-yl)-4(3H)-quinazolinone;

5-cyclopropyl-7-methoxy-2-(2-[(1S,4S)-2-oxa-5-azabicyclo[2.2.1]hept-5-ylmethyl]-7,8-dihydro[1,6]naphthyridin-6(5H)-yl)-4(3H)-quinazolinone or pharmaceutically acceptable salts or solvates thereof. Most preferred is 5-cyclopropyl-7-methoxy-2-(2-(4-morpholinylmethyl)-7,8-dihydro[1,6]naphthyridin-6(5H)-yl)-4(3H)-quinazolinone or pharmaceutically acceptable salts or solvates thereof; this compound is also referred to as Compound 1 herein.

Suitable α_{1A} and/or α_{1L} receptor antagonists also include compounds included in patent application WO 98/30560, preferably the compounds exemplified in WO 98/30560, even more preferably 4-amino-6,7-dimethoxy-2-(5-methanesulfonamido-1,2,3,4-tetrahydroisoquinol-2-yl)-5-(2-pyridyl)quinazoline, preferably a pharmaceutically acceptable salt or solvate thereof, most preferably the mesylate salt thereof.

Suitable α_{1A} and/or α_{1L} antagonists also include, for example, compounds in patent application WO 02/053558; KMD-3213/silodosin (Drugs of the Future (2001) 26, 553-560; EP600675); SNAP-7915 and other compounds disclosed in WO 00/35891; RWJ-69736 and other compounds disclosed in WO 99/42445; RS-100975 and other compounds disclosed in EP 949250; compounds disclosed in DiPardo et al ((2001) Bioorganic&Medicinal Chemistry Letters 11, 1959-1962). Other suitable α_{1A} and/or α_{1L} antagonists include tamsulosin, doxazosin, terazosin, or alfuzosin.

For the sake of clarity, a compound can combine high potency for both α_{1A} and α_{1L} adrenergic receptor, and the use of such a compound for the manufacture of a medicament for the treatment of FSD, in particular FSAD and/or FOD, is within the scope of the invention.

The invention relates to the use of an α_{1A} and/or α_{1L} adrenergic receptor antagonist for the treatment of FSD, preferably FSAD and/or FOD, alone, or in combination with one or more other agents such as

a phosphodiesterase (PDE) inhibitor, preferably a PDE5 inhibitor, such as sildenafil (see WO 03/051370 for a list of suitable compounds, as well as assays to identify suitable compounds), or

a neutral endopeptidase (NEP) inhibitor, such as compounds FV to FXI and F57 to F65 of EP1097719-A1 (see also WO 03/051370 for a list of suitable compounds), or

a central melanocortin agonist, preferably an MC-4 receptor agonist such as melanotan II, PT-14, or PT-141 (see WO 94/22460; U.S. Pat. No. 6,579,968); further examples can be found in WO 02/068387, WO 02/068388; WO 02/067869; WO 03/007949; WO 03/006604; WO 02/081443; WO 02/064091; WO 02059108; WO 02/059107; WO 02059095; WO 01/91752 and others, or

a dopamine receptor agonist, preferably a selective D3 receptor agonist, such as compounds disclosed in EP 0 899 267; see also the list of compounds in WO 03/051370, or

a neuropeptide Y (NPY) antagonist. e.g. as disclosed in WO 00/66578; see also list of compounds and assays to identify suitable compounds in WO 03/051370;

a serotonin receptor agonist, preferably a 5HT1A receptor agonist (including VML 670 (WO 02/074288) or flibanserin (EP526434)) or a 5HT2C-receptor agonist (e.g. m-CPP (m-chlorophenylpiperazine—commercially available from Sigma Aldrich Product number C-5554), compounds included in patent application WO 03/000666, preferably the compounds exemplified in WO 03/000666, even more preferably pharmaceutically acceptable salts or solvates thereof. Other 5-HT2C receptor agonists and antagonists can also be found, for example, in patent applications EP 863136, EP 657426, EP655440, EP 572863, WO 98/30548, WO 98/56768, WO 99/43647, WO 99/43647, WO 99/58490, WO 00/12475, WO 00/12482, WO 00/12502, WO 00/12510, WO 00/28993, WO 00/35922, WO 00/44737, WO 00/76984, or WO 02/074746. Another suitable 5-HT2C receptor agonist may be Ro-600175 (Jenk, F. et al (1998) Eur. J. Neuropharmacol. 8, 161-168; Dekeyne, A. et al (1999) Neuropharmacology 38, 415-423)). See also Bickerdike,M. J. (2003) Curr. Topics in Med Chem. 3, 885-897 for 5HT2C receptor agonists.

Preferably the patient will also be receiving Hormone Replacement Therapy (HRT), even more preferably HRT and additional androgen therapy. Agents used may include estrogen, estrogen and medroxyprogesterone or medroxyprogesterone acetate (MPA) (i.e. as a combination), or estrogen and methyl testosterone hormone replacement therapy agent (e.g. HRT especially Premarin, Cenestin, Oestrofeminal, Equin, Estrace, Estrofem, Elleste Solo, Estring, Eastraderm TTS, Eastraderm Matrix, Dermestril, Premphase, Preempro, Prempak, Premique, Estratest, Estratest HS, Tibolone). Agents for androgen therapy include testosterone replacement agent (including dehydroandrostendione), testosternone (Tostrelle), dihydrotestosterone or a testosterone implant.

Reference to an antagonist, an agonist or an inhibitor shall at all times be understood to include all active forms of such agents, including the free form thereof (e.g. the free and/or base form) and also all pharmaceutically acceptable salts, polymorphs, hydrates, silicates, stereo-isomers (e.g. diastereoisomers and enantiomers) and so forth. Active metabolites of any of the compounds, in any form, are also included.

Particular formulations of the compounds for either oral delivery or for topical application (creams, gels) are included in the invention. An intravaginal formulation comprising a compound or combination of compounds as defined herein, preferably a formulation which is a creme or a gel, is also included in the invention.

A method of enhancing sexual function of a female comprising administering an α_{1A} and/or an α_{1L} antagonist to a healthy female is a further aspect of the invention.

Yet a further aspect of the invention is a method of screening for compounds useful for treating FSD, preferably FSAD and/or FOD, comprising screening compounds for antagonist activity against α_{1A} and/or α_{1L} adrenergic receptor and selecting compounds with a Kᵢ of less than 100 nM, preferably with a Kᵢ of less than 10 nM, even more preferably with a Kᵢ of less than 1 nM in binding assays (see Example 2), or with a pA₂ of greater than 7, preferably a pA₂ of greater than 8, most preferably a pA₂ of greater than 9 in functional assays (see Example 3).

“Potency” as used herein is a measure of the concentration of a compound at which it is effective. The potency of a compound can be determined in a binding assay as described in Example 2, and potency in this context will refer to the Kᵢ of the compound, i.e. to the concentration of competing ligand in a competition assay which would occupy 50% of the receptors if no radioligand were present. The potency of a compound can also be determined in a functional assay such as contractile assays for different tissues expressing different a, receptor subtypes as described in Example 3. The potency in this case would refer to the pA₂ of the compound, i.e. the concentration of antagonist that produces a 2-fold shift in the agonist concentration-response curve.

“Selectivity” as used herein is a measure of the relative potency of a drug between two receptor subtypes for the same endogenous ligand. This can be determined, for example, in binding assays as described in Example 2, or in functional assays as described in Example 3.

### EXAMPLES

The invention will now be further described, by way of example, in which reference is made to the following Figure:

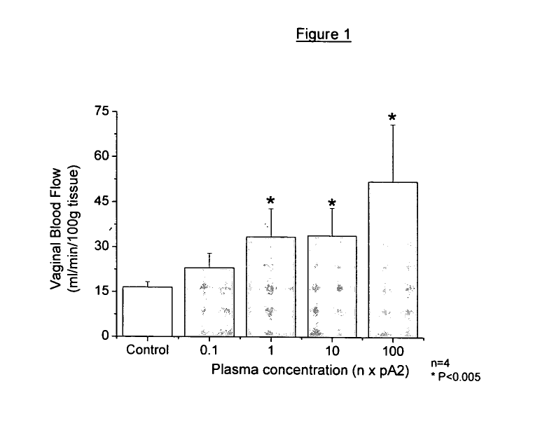

FIG. 1 is a bar chart, showing the plasma concentration of Compound 1, a potent and selective α_{1A} and/or α_{1L} adrenergic receptor antagonist, vs the vaginal blood flow in the anaesthetised rabbit model of female sexual arousal.

### Example 1

### Assay to Show Beneficial Effects of Compounds and Combination of Compounds in FSAD

We have developed an animal model that mimics the physiological arousal response observed during female sexual arousal and directly reflects the clinical data obtained in human volunteers. The model uses Laser Doppler technologies to record small changes in vaginal and clitoral blood flow induced by pelvic nerve stimulation or vasoactive neurotransmitters. During sexual arousal, there is an increase in genital blood flow resulting from increased innervation from the pelvic nerve. The pelvic nerve-stimulated increase in vaginal and clitoral blood flow, observed in the animal model, represents the endogenous vascular effects observed during female sexual arousal. Therefore this model can be used to firstly, identify the mechanisms involved in the regulation of vaginal and clitoral blood flow and secondly, to validate novel approaches for the enhancement of genital blood flow.

(a) The Experimental Details are Described in European patent application EP 1 097 719 A1, Paragraphs [0495]-[0500]

(b) Infusion of Test Compound to Achieve Free Plasma Concentration Equivalent to 0.1 to 100 Times the pA₂ α_{1A} and/or α_{1L} Adrenergic Receptors.

To achieve steady state plasma concentrations of the test compound, a loading dose was administered followed by infusion of a steady state dose. The test compound was made up in acidified saline (5% 1M HCl in saline) and details of the infusion protocols are given in the Table below. Test compound and vehicle controls were infused at the same rate. The test compound was infused for 15 minutes prior to pelvic nerve stimulation.

| Target Plasma | | |
|---|---|---|
| Concentration | Loading Infusion | Maintenance Infusion |
| (multiple of pA₂) | (μg/kg/min i.v.) | (μg/kg/min/min i.v.) |
| 0.1 | 0.84 | 0.003 |
| 1 | 8.4 | 0.01 |
| 10 | 84 | 0.03 |
| 100 | 840 | 0.1 |

(c) Statistical Analysis

All data are reported as mean±s.e.m. (Standard error of the mean). Significant changes were identified using Student's t-tests (independent).

A major cause of FSAD is decreased genital blood flow and this manifests itself as reduced vaginal, labial and clitoral engorgement. Treatment of women with FSAD is achievable by restoration of the normal sexual arousal response. This can be achieved by enhancing genital blood flow.

FIG. 1 shows effect of administering a selective α_{1A} and/or α_{1L} adrenergic receptor antagonist on the genital blood flow in a rabbit. Compound 1, a potent and selective α_{1A} and/or α_{1L} adrenergic receptor antagonist, enhanced pelvic nerve stimulated (PNS) increases in genital blood flow in the anaesthetised rabbit model of sexual arousal. Repetitive PNS at 15 minute intervals induced reproducible increases in genital blood flow (Open Bar). Administration of a selective α_{1A} and/or α_{1L} adrenergic receptor antagonist (Grey bars) dose dependently enhanced the peak increase in vaginal blood flow induced by submaximal stimulation frequencies (eg 4 Hz) compared to increases observed during time matched control stimulations or vehicle controls (Open bar). The percentage increase over basal stimulations are showin in table 1 below. Clitoral blood flow was also increased (data not shown). No significant cardiovascular effects were observed. Data expressed as mean±sem (n=4); all changes were monitored using laser Doppler technologies.

TABLE 1

| | Potentiation of pelvic nerve |
|---|---|
| | stimulated increases in |
| Free plasma concentration | vaginal blood flow |
| (0.1 to 100 times the pA₂ α_{1A} | (% increase over basal |
| and/or α_{1L} adrenergic receptors) | increase) |
| 0.1 × pA2 | 39% |
| 1 × pA2 | 102% |
| 10 × pA2 | 104% |
| 100 × pA2 | 213% |

Enhancement of genital blood flow by potent and selective α_{1A} and/or α_{1L} adrenergic receptor antagonists will be useful in the treatment of FSAD. By enhancing genital blood flow they will potentiate vaginal engorgement/lubrication and clitoral engorgement/sensitivity. This will have the overall effect of restoring or potentiating the normal arousal response with the absence of significant cardiovascular side effects.

### Example 2

### Binding Assay to α₁ receptor subtypes

Binding assays to the α₁ receptor subtypes can be carried out by standard techniques, well known to the skilled person. In brief, transfected cells expressing human or mammalian α₁ adrenergic receptor subtypes (Schwinn, D. A. (1995) J. Pharmacol. Exp. Ther. 272, 134-142; Schwinn, D. A. et al (1990) J. Biol. Chem. 265, 8183-8189; Cotecchia, S. et al (1988) Proc. Natl. Acad. Sci. USA 85, 7159-7163; Lomasney, J. W. et al (1991) J. Biol. Chem. 266, 6365-6369) are scraped into 50 mM Tris-HCl, pH7.5, and lysed by sonication. The cell lysates are centrifuged at 1000 rpm for 5 min at 4° C. The supernatant is centrifuged at 30,000×g for 20 min at 4° C. The pellet is resuspended in 50 mM Tris-HCl. Binding of the α₁ antagonist [³H]-prazosin (0.3 nM final concentration) is carried out at room temperature for 30 minutes. Non-specific binding is determined in the presence of 10 μM phentolamine. The reaction is stopped by filtration through GF/B filters presoaked in 50 mM Tris/HCl, 0.5% polyethylenimine PEI (w/v), and the radioactivity on the filters measured by scintillation counting. Inhibition experiments are carried out with a range of concentration of test compound; the results are analysed using non-linear regression curve fitting computer programs for obtaining Kᵢ values.

### Example 3

### Functional Assays—Contractile Responses in Various Tissues

(a) Contractile Responses of Rabbit Aorta (α_{1L} Receptor)

A single rabbit aorta was cleaned of connective tissue, cut into rings ˜3 mm in length, then denuded of epithelium by rubbing very gently with a probe. The lengths of tissue are then mounted in the 5 ml organ baths, which contain the modified Krebs of the following composition (mM): NaCl (119), KCl (4.7), CaCl₂ (2.5), KH₂PO₄ (1.2), MgSO₄ (1.2), NaHCO₃ (25), glucose (11), and gassed with 95% O₂/5% CO₂. The tissues are placed under ˜1.5 g tension, and are left to equilibrate for ˜60 minutes on a pump speed of ˜5ml\minute, adjusting the tension to 1-1.5 g if necessary after 15 and 45 minutes. A 1M stock solution (1×10⁻³M bath conc) of methoxamine in water was made and 1:10 dilutions made using the same diluent. A sensitising dose of 120 mM KCl (bath concentration) was added to each bath. After the maximum response had been reached (usually about 6-8 minutes), the tissues are washed with Krebs for 60 minutes, pump speed at ˜2.97 ml/min.

A cumulative dose response curve was constructed, bath concentrations of methoxamine being 1×10⁻⁷M to a maximum of 3×10⁻⁴M. Each dose was allowed to exert its maximum effect before the next dose was added (6-8 mins). On completion of this curve, the tissues were washed, (pump speed ˜10 ml/min for 10 minutes, 2.97 ml/min for 50 minutes) until the tissues were stable at baseline tension.

The compound under investigation was made up to a stock concentration of 1 mM in 100% DMSO. Three chosen concentrations for a pA₂ estimation were then made up in DMSO, and 5 μl of each concentration added in duplicate to the tissues, with a vehicle control (DMSO). The tissues were left in the presence of compound or vehicle for 60 minutes before a second CDRC to methoxamine was constructed up to a maximum of 3×10⁻³M.

The data was captured on ADA analysis in-vitro software, which expresses the readings as a % of the maximum response of the control curve, draws control and test compound dose response curves, and calculates a EC₅₀ and then dose ratio (DR), the ratio between control and treatment curve EC₅₀, for each treatment. The results are reported as pA₂.

The pA₂ was plotted on a Schild analysis. ie y axis=log (DR*−1); x axis=−log antagonist concentration

where

$${DR}^{*} = \frac{dose ratio compound}{dose ratio control}$$

NB. If the value of (DR*−1) was less than or equal to 2, the result could not be used for a pA₂ estimation. The control curves must not have shifted by more than 2.5.

(b) Contractile Responses of Rat Vas Deferens (α_{1A} Receptor)

Rat vas deferens were cleaned of associated blood vessels and connective tissue, and the epidydimal (thinner) end cut to ˜15 mm in length. The lengths of tissue were mounted in 5 ml organ baths, which contain the modified Krebs of the following composition (mM): NaCl (119), KCl (4.7), CaCl₂ (2.5), KH₂PO₄ (1.2), MgSO₄ (1.2), NaHCO₃ (25), glucose (11), and gassed with 95% O₂/5% CO₂. The tissues were placed under to ˜1 g tension, and left to equilibrate for ˜60 minutes on a pump speed of ˜5ml\minute. The tension is adjusted during this period to ˜1 g to stabilise the resting tension. A 0.1M stock solution of noradrenaline (NA) was made in dilute ascorbic acid solution (0.1 mg/ml), and 1:10 dilutions made using the same diluent. A sensitising dose of 1×10⁻⁴M noradrenaline, was added to each bath. After the maximum response had been reached (˜1 minute), the tissues were washed with Krebs for 1 hour, pump speed at ˜2.97 ml/min. A control non-cumulative dose response curve (NCDRC) is constructed, using 0.5 log dose increments, bath concentrations of NA being: 1×10⁻⁸M to 3×10⁻⁵M.

Following each response the tissues were washed at 5 ml/min for 5 minutes prior to the next concentration being added. All reading were for 90 seconds reading the “area under the curve” for each response. On completion of this curve, the tissues were washed (pump speed max for 5 seconds, 2.97 ml/min for 60 minutes).

The compound under investigation is made up to a stock concentration of 1 mM in 100% DMSO. Three chosen concentrations for a pA₂ estimation were then made up in 1 litre of the modified Krebs, and perfused over tissues in duplicate, with a Krebs+vehicle (DMSO) for control, for 60 minutes, pump speed 2.97 ml/min. A second NCDRC to NA was constructed (1×10⁻⁸ to 3×10⁻³M) in all tissues as described above, using the relevant antagonist-Krebs solution for washes between responses.

The data was captured on ADA analysis in-vitro software, which expresses the readings as a % of the maximum response of the control curve, draws control and test compound dose response curves, and calculates a EC₅₀ and then dose ratio (DR), the ratio between control and treatment curve EC₅₀, for each treatment. The results are reported as pA₂.

The pA₂ was plotted on a Schild analysis. ie y axis=log (DR*−1); x axis=−log antagonist concentration

where

$${DR}^{*} = \frac{dose ratio compound}{dose ratio control}$$

NB. If the value of (DR*−1) was less than or equal to 2, the result could not be used for a pA₂ estimation. The control curves must not have shifted by more than 2.5.

(c) Contractile Responses of Rat Aorta (α_{1D} Receptor)

Rat aortae were cleaned of connective tissue, cut to −3 mm in length, then denuded of epithelium by rubbing very gently with a probe. The lengths of tissue are then mounted in the 5 ml organ baths, which contain the modified Krebs of the following composition (mM): NaCl (119), KCl (4.7), CaCl₂ (2.5), KH₂PO₄ (1.2), MgSO₄ (1.2), NaHCO₃ (25), glucose (11), and gassed with 95% O₂/5% CO_{2.} The tissues were placed under ˜1 g tension, and were left to equilibrate for ˜60 minutes on a pump speed of ˜5 ml\minute, adjusting the tension to 1-1.5 g if necessary after 15 and 45 minutes. A 0.1M stock solution of noradrenaline (NA) was made in dilute ascorbic acid solution (0.1 mg/ml), and 1:10 dilutions made using the same diluent. A sensitising dose of 1×10⁻⁶M noradrenaline (bath concentration) was added to each bath. After the maximum response had been reached (usually about 3-4 minutes), the tissues were washed with Krebs for 30 minutes, pump speed at ˜2.97 ml/min.

A cumulative dose response curve was constructed, bath concentrations of NA being 1×10⁻⁹M to a maximum of 1×10⁻⁶M. Each dose was allowed to exert its maximum effect before the next dose was added (2-4 mins). On completion of this curve, the tissues were washed, (pump speed ˜10 ml/min for 10 minutes, 2.97 ml/min for 20 minutes) until the tissues were stable at baseline tension.

The compound under investigation was made up to a stock concentration of 1 mM in 100% DMSO. Three chosen concentrations for a pA₂ estimation were made up in DMSO, and 5 μl of each concentration added in duplicate to the tissues, with a vehicle control (DMSO). The tissues were left in the presence of compound or vehicle for 60 minutes.

A second CDRC to NA was constructed as described previously, up to a maximum of 3×10⁻³M.

The data was captured on ADA analysis in-vitro software, which expresses the readings as a % of the maximum response of the control curve, draws control and test compound dose response curves, and calculates a EC₅₀ and then dose ratio (DR), the ratio between control and treatment curve EC₅₀, for each treatment. The results are reported as pA₂.

The pA₂ was plotted on a Schild analysis. ie y axis=log (DR*−1); x axis=−log antagonist concentration

where

$${DR}^{*} = \frac{dose ratio compound}{dose ratio control}$$

NB. If the value of (DR*−1) was less than or equal to 2, the result could not be used for a pA₂ estimation. The control curves must not have shifted by more than 2.5.

(d) Contractile Responses of Rat Spleen (α_{1B} Receptor)

Rat spleens were cleaned of connective tissue, the ends removed and cut longitudinally in two. The lengths of tissue were then mounted in the 5 ml organ baths, which contain modified Krebs of the following composition (mM): NaCl (119), KCl (4.7), CaCl₂ (2.5), KH₂PO₄ (1.2), MgSO₄ (1.2), NaHCO₃ (25), glucose (11), and gassed with 95% O₂/5% CO₂. The tissues were placed under 1 g tension, and were left to equilibrate for ˜90 minutes on a pump speed of ˜3 ml\minute. Tissue tension was not adjusted during this period. Tissue tension equilibrated to ˜500-700 mg. A 0.1M stock solution of noradrenaline (NA) was made in dilute ascorbic acid solution (0.1 mg/ml), and 1:10 dilutions made using the same diluent. A sensitising dose of 1×10⁻⁴M noradrenaline, was added to each bath. After the maximum response had been reached (˜6 minutes), the tissues were washed with Krebs for 90 minutes, pump speed ˜3 ml/min. A second sensitising dose of 1×10⁴M noradrenaline was then added as above and upon reaching the maximum response, the tissues were washed with Krebs as above. A cumulative dose response curve (CDRC) was constructed, bath concentrations of phenylephrine being: 1×10⁻⁸M to 3×10⁻⁴M.

The compound under investigation was made up to a stock concentration of 1 mM in 100% DMSO. Three chosen concentrations for a pA₂ estimation were made up in DMSO, and 5 μl of each concentration added in duplicate to the tissues, with a vehicle control (DMSO). The tissues were left in the presence of compound or vehicle for 60 minutes. A second CDRC to NA was constructed as described previously, up to a maximum of 3×10⁻³M.

The data was captured on ADA analysis in-vitro software, which expresses the readings as a % of the maximum response of the control curve, draws control and test compound dose response curves, and calculates a EC₅₀ and then dose ratio (DR), the ratio between control and treatment curve EC₅₀, for each treatment. The results are reported as pA₂.

The pA₂ was plotted on a Schild analysis. ie y axis=log (DR*−1); x axis=−log antagonist concentration

where

$${DR}^{*} = \frac{dose ratio compound}{dose ratio control}$$

NB. If the value of (DR*−1) was less than or equal to 2, the result could not be used for a pA₂ estimation. The control curves must not have shifted by more than 2.5.

(e) Contractile Responses of Human Prostate (α_{1L} Receptor)

Prostatic tissue was cut into longitudinal strips (approximately 3×2×10 mm) and suspended in organ baths under a resting tension of 1 g in Krebs Ringer bicarbonate of the following composition (mM): NaCl (119), KCl (4.7), CaCl₂ (2.5), KH₂PO₄ (1.2), MgSO₄ (1.2), NaHCO₃ (25), glucose (11), and gassed with 95% O₂/5% CO₂. The solution also contained 10 mM cocaine and 10 mM corticosterone. Tissues were sensitised using a full concentration-response curve to (−)-noradrenaline (100 nM to 30 μM) and then washed over a 60 minute period. Isometric contractions were obtained in response to cumulative additions of (−)-noradrenaline to obtain control curves in all tissues. A further curve was then generated in the presence or absence of antagonist (incubated for 2 hours). Antagonist affinity estimates (pA₂) were determined using a single concentration of competing antagonist, pA₂=−log [A]/(DR−1) where the dose ratio (DR), relative to corresponding controls, was produced by a single concentration of antagonist [A], assuming competitive antagonism and Schild regression close to unity.

### Example 4

### Clinical Study

A clinical study with a suitable α_{1L} antagonist, e.g. Compound 1 or 4-Amino-6,7-dimethoxy-2-(5-methanesulfonamido-1,2,3,4-tetrahydroisoquinol-2-yl)-5-(2-pyridyl)quinazoline (mesylate salt), can be carried out following protocols similar to those for published studies with sildenafil (Caruso, S. et al (2001) BJOG 108, 623-628; Berman, J R et al (2001) J Sex Marital Ther. 27, 411-420).

Briefly, women with FSAD are given a suitable dose of the compound or a placebo. The skilled person will be able to determine a suitable dose for the compound to be used; for the two compounds mentioned above, a dose range of 0.1 to 50 mg could be used. Evaluation of the efficacy of the treatment can be carried out by physiologic measurements in the clinic (e.g. measuring clitoral, labial (vestibular bulb), urethral, and vaginal arterial-peak systolic velocity and end diastolic velocity using duplex Doppler ultrasonography; vaginal pH using a digital pH meter, maximum intravaginal pressure/volume changes using commercially available compliance balloons, vibratory perception thresholds recorded from the clitoris and the mucosal aspects of the right and left labia minora using a standard biothesiometer), as well as by questionnaire, assessing and quantifying, e.g. subjective arousal, orgasm, enjoyment, sexual frequency, and number of sexual fantasies.

1. A method of treating FSD which method comprises administering to a patient in need of such treatment an effective amount of a compound that is an α_{1A} and/or an α_{1L} adrenergic receptor antagonist.

2. A method of claim 1, wherein the α_{1A} and/or an α_{1L} adrenergic receptor antagonist has a Kᵢ in a binding assay of less than 100 nM, or a pA₂ greater than 7 in a functional assay.

3. A method of claim 1, wherein the α_{1A} and/or an α_{1L} adrenergic receptor antagonist has a Kᵢ in a binding assay of less than 10 nM, or a pA₂ greater than 8 in a functional assay.

4. A method of claim 1, wherein the α_{1A} and/or an α_{1L} adrenergic receptor antagonist has a Kᵢ in a binding assay of less than 1 nM, or a pA₂ greater than 9 in a functional assay.

5. A method of claim 1, wherein the compound is a selective α_{1L} and/or a selective α_{1A} adrenergic receptor antagonist.

6. A method of claim 5, wherein the compound is more than 10-fold selective for α_{1L} and/or α_{1A} receptor over α_{1B} receptor.

7. A method of claim 5, wherein the compound is more than 10-fold selective for α_{1L} and/or α_{1A} receptor over α_{1D} receptor.

8. A method of claim 5, wherein the compound is more than 10-fold selective for α_{1L} and/or α_{1A} receptor over α_{1D} and α_{1B} receptors.

9. A method of claim 1, wherein FSD is FSAD and/or FOD.

10. A method of claim 1, wherein the compound is a compound of formula (I): 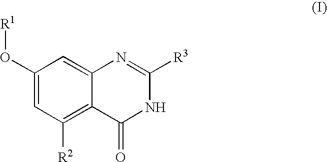
or a pharmaceutically acceptable salt or solvate thereof, wherein:
R¹ represents C₁₋₄ alkyl;
R² represents C₃₋₆ cycloalkyl;
R³ represents a bicyclic group of the formula 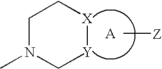
wherein X and Y are selected from C and N, provided that at least one is C;
Ring A together with X and Y represents a 5- or 6-membered aromatic ring containing 0, 1, 2 or 3 nitrogen atoms in the ring;
Z is selected from H, and LR⁴;
L represents a direct link, C₁₋₄ alkylene or C₁₋₄ alkoxyalkylene;
R⁴ represents H, NR⁵R⁶, C₃₋₆ cycloalkyl, OR⁷ or Het¹;
R⁵ and R⁶ are independently selected from H, C₃₋₆ cycloalkyl and C₁₋₄ alkyl optionally substituted with OR⁸;
R⁷ is selected from H, C₁₋₄ alkyl, C₁₋₄ alkoxyalkyl, C₃₋₆cycloalkyl, Het² and C₁₋₄alkyl-Het³;
R⁸ is H or C₁₋₄ alkyl;
Het¹, Het² and Het³ independently represent a 4 to 7 membered saturated heterocyclic group which may be mono- or bi-cyclic and which contains one or more heteroatoms selected from N, O or S, optionally substituted with OR⁹ and/or C₁₋₄ alkyl optionally substituted by OR⁹;
R⁹ is H or C₁₋₄ alkyl.

11. A method of claim 10, wherein the compound is selected from:
5-cyclopropyl-7-methoxy-2-(2-([dimethylamino]methyl)-7,8-dihydro[1,6]naphthyridin-6(5H)-yl)-4(3H)-quinazolinone;
5-cyclopropyl-7-methoxy-2-(2-(1-pyrrolidinylmethyl)-7,8-dihydro[1,6]naphthyridin-6(5H)-yl)-4(3H)-quinazolinone;
5-cyclopropyl-7-methoxy-2-(2-(4-morpholinylmethyl)-7,8-dihydro[1,6]naphthyridin-6(5H)-yl)-4(3H)-quinazolinone;
5-cyclopropyl-7-methoxy-2-(5-([dimethylamino]methyl)-3,4-dihydro[2,6]naphthyridin-2(1H)-yl)-4(3H)-quinazolinone;
5-cyclopropyl-7-methoxy-2-(5-(1-pyrrolidinylmethyl)-3,4-dihydro[2,6]naphthyridin-2(1H)-yl)-4(3H)-quinazolinone;
5-cyclopropyl-7-methoxy-2-(5-(1-piperidinylmethyl)-3,4-dihydro[2,6]naphthyridin-2(1H)-yl)-4(3H)-quinazolinone;
5-cyclopropyl-7-methoxy-2-(5-(4-morpholinylmethyl)-3,4-dihydro[2,6]naphthyridin-2(1H)-yl)-4(3H)-quinazolinone;
5-cyclopropyl-7-methoxy-2-(5-[(1S,4S)-2-oxa-5-azabicyclo[2.2.1]hept-5-ylmethyl]-3,4-dihydro[2,6]naphthyridin-2(1H)-yl)-4(3H)-quinazolinone;
5-cyclopropyl-7-methoxy-2-(2-[(1S,4S)-2-oxa-5-azabicyclo[2.2.1]hept-5-ylmethyl]-7,8-dihydro[1,6]naphthyridin-6(5H)-yl)-4(3H)-quinazolinone, or pharmaceutically acceptable salts or solvates thereof.

12. A method of claim 5, wherein the compound is 4-amino-6,7-dimethoxy-2-(5-methanesulfonamido-1,2,3,4-tetrahydroisoquinol-2-yl)-5-(2-pyridyl)quinazoline, or a pharmaceutically acceptable salt or solvent thereof.

13. A method of claim 1, wherein the compound is selected from tamsulosin, doxazosin, terazosin, alfuzosin, or silodosin.

14. An intravaginal formulation comprising a compound as defined in claim 1.

15. A formulation as claimed in claim 14, which is a cream or a gel.

16. A method of enhancing sexual function in a female which method comprises administering to a healthy female an α_{1A} and/or an α_{1L} adrenergic receptor antagonist.

17. A method of screening for compounds useful for treating FSD, which method comprises screening compounds for antagonist activity against α_{1A} and/or α_{1L} adrenergic receptor, and selecting compounds with an Kᵢ of less than 100 nM, or a pA₂ greater than 7.

18. A method of treating or preventing FSD which method comprises administration of a combination comprising one or more α_{1A} and/or an α_{1L} adrenergic receptor antagonists, and one or more of the following auxiliary agents:
(a) A PDE5 inhibitor;
(b) A neutral endopeptidase (NEP) inhibitor;
(c) A Dopamine D3 receptor agonist;
(d) A 5HT1A receptor agonist or a 5HT2C receptor agonist;
(e) Agents used for hormone replacement therapy (HRT); and
(f) Agents used in combination for HRT and additional androgen therapy.
